(19) United States
(12) Patent Application Publication
(10) Pub. No.: US 2022/0399083 A1
(43) Pub. Date: Dec. 15, 2022

(54) Method for finding an optimal quantum state minimizing the energy of a Hamiltonian operator with a quantum processor by using a VQE method, determining a quantum state of a chemical compound, and determining physical quantum properties of materials

(71) Applicant: BULL SAS, LES CLAYES SOUS BOIS (FR)

(72) Inventors: Thomas AYRAL, Versailles (FR); Pauline BESSERVE, Versailles (FR)

(73) Assignee: BULL SAS, LES CLAYES SOUS BOIS (FR)

(21) Appl. No.: 17/835,328

(22) Filed: Jun. 8, 2022

(30) Foreign Application Priority Data
Jun. 9, 2021 (EP) 21305788.8

(51) Int. Cl. G16C 10/00; G06N 10/60; G06N 10/20

(52) U.S. Cl. CPC G16C 10/00; G06N 10/60; G06N 10/20

(57) ABSTRACT

A method for finding an optimal quantum state minimizing the energy of a Hamiltonian operator with a quantum processor and a classical processor comprising a quantum circuit for producing trial quantum states for the Hamiltonian operator and parametric quantum gates with associated parameters, by using a VQE method, the method comprising: providing the Hamiltonian operator in an orbital basis and iteratively, until a predefined stopping criterion is satisfied: (i) applying the VQE method to find optimized values for the parameters that yield an intermediate optimal quantum state which minimizes the energy of the Hamiltonian operator, (ii) computing a one particle reduced density matrix (1-RDM) based on the intermediate optimal quantum state, (iii) determining an updated orbital basis in which the 1-RDM is diagonal, and an associated transformation matrix, and (iv) modifying the Hamiltonian operator with the transformation matrix; and then returning, as the optimal quantum state the intermediate optimal quantum state that minimizes the most the energy.

### CROSS-REFERENCE TO RELATED APPLICATIONS

This application claims priority to EP 21305788.8, filed on Jun. 9, 2021, which is incorporated by reference herein in its entirety.

### TECHNICAL FIELD

This disclosure pertains to the field of methods for finding an optimal quantum state minimizing the energy of a Hamiltonian operator with a quantum processor by using a Variational Quantum Eigensolver, VQE, method, which can be used for determining a quantum state of a chemical compound or for determining physical quantum properties of materials.

### BACKGROUND

Currently, the performance of the quantum programs that can be executed on quantum processors is severely limited by their duration and complexity, in part due to the quantum noise affecting the quantum processors and the important depths of the quantum circuits implementing the quantum program. In particular, quantum programs used to compute the minimum of a cost function need to be tailored to respect the noise constraints to avoid a large accuracy loss. Existing methods require quantum circuit depths that are usually incompatible with the quantum processors noise constraints leading to poor accuracies in the determination of the minimum of a cost function.

Some methods have been developed in order to compute the minimum of a cost function, expressed as the expectation value of a Hamiltonian operator over a variational state, by using a variational method known as Variational Quantum Eigensolver (VQE) method. Generally speaking, this method is used to estimate the quantum ground state of a given Hamiltonian operator. The computing of the VQE method starts from a trial quantum state constructed using a quantum circuit defined by a set of parameters such as rotation gates' angles. The energy associated with the Hamiltonian operator is computed for this given instance of the trial circuit. Then, a classical minimizer proposes new values for the circuit's parameters. The process goes on with updated parameters being proposed by the classical minimizer based on the energy evaluations provided by the quantum computer until hopefully the minimal energy is returned. For this method to succeed, the trial quantum circuit must be expressive enough (typically, the more parametrized gates the quantum circuit encompasses, the more expressive it is), the minimization procedure must be feasible (the more parameters in the set of parameters of the quantum circuit, the harder the minimization) and the trial quantum state preparation and energy measurement must be accurate. However, on noisy quantum processors, the actual trial quantum state that is prepared departs from the expected perfect quantum state due to quantum noise, such that the measurement of its energy may also be altered, resulting in speed and accuracy problems in the convergence of the VQE methods.

Another method called PermVQE has been developed in the non-patent literature Correlation-Informed Permutation of Qubits for Reducing Ansatz Depth in VQE, Tkachenko et al arXiv preprint arXiv:2009.04996, 2020. In this paper, it is proposed to add an optimization loop to the VQE method that permutes qubits in order to solve for the qubit Hamiltonian that minimizes long-range correlations in the quantum ground state. The qubits are originally represented in an initial orbital basis, which is permuted in the optimization loop by the permutation of the qubits. The choice of permutations is based on mutual information, which is a measure of interaction between electrons in spin-orbitals. Encoding strongly interacting spin-orbitals into proximal qubits on a quantum processor with a nearest-neighbor connectivity (as is the case for e.g superconducting processors) naturally reduces the circuit depth needed to prepare the trial quantum state, as it does not require the insertion of additional SWAP gates (or other qubit routing methods) to satisfy the connectivity constraints.

It is also known from the numerous classical quantum chemistry and condensed-matter approaches how to exploit the freedom in selecting the basis in which the qubits are represented for improving the speed and/or accuracy of their algorithms. The prior art Quantum Theory of Many-Particle Systems. I. Physical Interpretations by Means of Density Matrices, Natural Spin-Orbitals, and Convergence Problems in the Method of Configurational Interaction, Physical Review volume 97 number 6, Mar. 15, 1955, Per-Olov Lödwin, describes the concept of natural orbital basis to designate the basis in which the representation of a quantum state is the simplest, i.e. the basis that necessitates the least number of Slater determinants to represent the quantum state. In quantum mechanics, a Slater determinant is an expression that describes the wave function of a non-interacting multi-fermionic system. It satisfies anti-symmetry requirements, and consequently the Pauli principle, by changing sign upon exchange of two fermions. What is interesting from a quantum computing standpoint is that a Slater determinant is a state of the computational basis, i.e. a state that can be described with a single state on the qubits. As the number of Slater determinants in the representation of a quantum state dictates the complexity of the quantum circuit required to prepare it, minimizing it is crucial. However, the method is applicable only with classical processors, i.e. non-quantum processors. Moreover, the natural orbital basis one is interested in going to depends on the quantum ground state, which is not known since it is precisely what one is searching for. The natural orbital basis can thus be computed only a posteriori.

### SUMMARY

The invention at least partially remedies the shortcuts of the prior arts and optimizes the minimization of the energy associated with a Hamiltonian operator with a quantum processor.

According to a first aspect, it is proposed a method for finding an optimal quantum state minimizing the energy associated with a Hamiltonian operator with a quantum processor by using a Variational Quantum Eigensolver, VQE, method, the quantum processor comprising a predetermined quantum circuit for producing trial quantum states for the Hamiltonian operator, said predetermined quantum circuit comprising at least parametric quantum gates associated with one or more parameters to be optimized, the method comprising:

providing the Hamiltonian operator in an orbital basis and iteratively, until a predefined stopping criterion is satisfied:
applying the VQE method to find optimized values for at least some of the parameters associated with the parametric quantum gates of the predetermined quantum circuit that yield an intermediate optimal quantum state which minimizes the energy associated with the Hamiltonian operator,
computing a one particle reduced density matrix based on the intermediate optimal quantum state,
determining an updated orbital basis in which the one particle reduced density matrix is diagonal, and an associated transformation matrix,
modifying the Hamiltonian operator by using the transformation matrix,

wherein, when the predefined stopping criterion is satisfied, the method further comprises returning, as the optimal quantum state minimizing the energy associated with the Hamiltonian operator, the intermediate quantum state which minimizes the most the energy associated with the Hamiltonian operator.

Therefore, the VQE method is performed iteratively with an additional phase during which the orbital basis in which the Hamiltonian operator is provided gets updated based on the intermediate optimal quantum state determined by the VQE method. By iteratively determining an intermediate optimal quantum state and updating the orbital basis, the orbital basis progressively approaches the natural orbital basis, thereby progressively enhancing the expressivity of the quantum circuit used for preparing trial quantum states. The transformation of the minimization problem to the natural orbital basis endows the method with a guarantee that the minimization of the energy associated with the Hamiltonian operator is in fine performed in the most economical basis, enabling to compromise between the size of the quantum circuit used to prepare trial quantum states and its expressivity in the orbital basis.

The method enables for quantum circuits, preferably of low-depth, that may not be expressive enough in the initial orbital basis, to become more and more expressive as the method converges to the natural orbital basis. At the same time, since these predetermined quantum circuits may be of limited depth, they are robust to noise, thereby enhancing the accuracy of the minimization.

In another aspect, it is proposed a method for determining a quantum state of a chemical compound, such as a molecule, comprising a method for finding an optimal quantum state minimizing the energy associated with a Hamiltonian operator with a quantum processor, wherein an expectation value of the Hamiltonian operator over a given quantum state corresponds to the energy of said quantum state.

In another aspect, it is proposed a method for determining physical properties of materials comprising a method for finding an optimal quantum state by minimizing the energy associated with a Hamiltonian operator with a quantum processor.

The following features, can be optionally implemented, separately or in combination one with the others:

the step of applying the VQE method is an iterative scheme in which the quantum processor is used in conjunction with a classical processor, the quantum processor preparing a trial quantum state for the Hamiltonian operator and performing measurements representative of the energy associated with the Hamiltonian operator for said trial quantum state, and the classical processor updating values of the parameters of the parametric quantum gates of the predetermined quantum circuit based on the measurements performed by the quantum processor, the iterative scheme being executed until a second predefined stopping criterion is satisfied, the VQE method returning the optimized values of the parameters;

the predetermined quantum circuit is a product quantum circuit comprising only one-qubit quantum gates in the form of rotations, or a quantum circuit comprising fSim quantum gates or a Low-Depth Circuit Ansatz, LDCA, quantum circuit;

a physical quantum state is encoded into a qubit state by means of the Jordan-Wigner transformation from which the Hamiltonian operator is decomposed accordingly in terms of qubit observables

the method is applied on a Hubbard model for which the Hamiltonian operator is provided;

the Hubbard model is a Hubbard model at half-filling;

the Hamiltonian operator is a second-quantized Hamiltonian;

the Hamiltonian operator represents an energy of a molecule, the optimal quantum state corresponding to the eigenvector associated the lowest eigenvalue;

the molecule is a H2, LiH and/or H2O molecule;

the number of qubits in the predetermined quantum circuit corresponds to a number of spin-orbitals used to describe the molecule;

the predefined stopping criterion is a maximum number of iterations and/or a minimum change of a variance between the minimums of the energy associated with the Hamiltonian operator obtained after two consecutive iterations;

the parametric quantum gates comprise rotation quantum gates, and wherein the parameters associated with said rotation quantum gates comprise values of angles.

### BRIEF DESCRIPTION OF DRAWINGS

Other features, details and advantages will be shown in the following detailed description and on the figures, on which:

FIG. 1 is a flow diagram of the method for finding a minimum of a cost function with a noisy quantum processor.

FIG. 2 is a flow chart illustrating some of the main steps of the method of FIG. 1.

FIG. 3A illustrates an example of a quantum circuit comprising a product of rotations.

FIG. 3B illustrates an example of an fSim quantum circuit comprising fSim quantum gates

FIG. 3C illustrates an example of a LDCA quantum circuit.

FIG. 4A illustrates the performance of the method applied on the Hubbard model at half-filling on two sites, with μ=U/2 and U=0

FIG. 4B illustrates the performance of the method applied on the Hubbard model at half-filling on two sites, with μ=U/2 and U=1.

FIG. 5A illustrates the performance of the method applied on the Hubbard model at half-filling on four sites, with μ=U/2 and U=0.

FIG. 5B illustrates the performance of the method applied on the Hubbard model at half-filling on four sites, with μ=U/2 and U=1.

FIG. 6A illustrates the performance of the method applied on the Hubbard model at half-filling on four sites, with μ=U/2 and U=0, on a noisy processor with a depolarizing noise of magnitude 0.001 for the 1-qubit quantum gates and 0.01 for the 2-qubits quantum gates

FIG. 6B illustrates the performance of the method applied on the Hubbard model at half-filling on four sites, with μ=U/2 and U=1, on a noisy processor with a depolarizing noise of magnitude 0.001 for the 1-qubit quantum gates and 0.01 for the 2-qubits quantum gates.

FIG. 7A illustrates the performance of the method applied on the Hubbard model at half-filling on four sites, with μ=U/2 and U=0, on a noisy processor a depolarizing noise of magnitude 0.005 for the 1-qubit quantum gates and 0.05 for the 2-qubits quantum gates

FIG. 7B illustrates the performance of the method applied on the Hubbard model at half-filling on four sites, with μ=U/2 and U=1, on a noisy processor a depolarizing noise of magnitude 0.001 for the 1-qubit quantum gate and 0.01 for the 2-qubits quantum gates.

### DETAILED DESCRIPTION

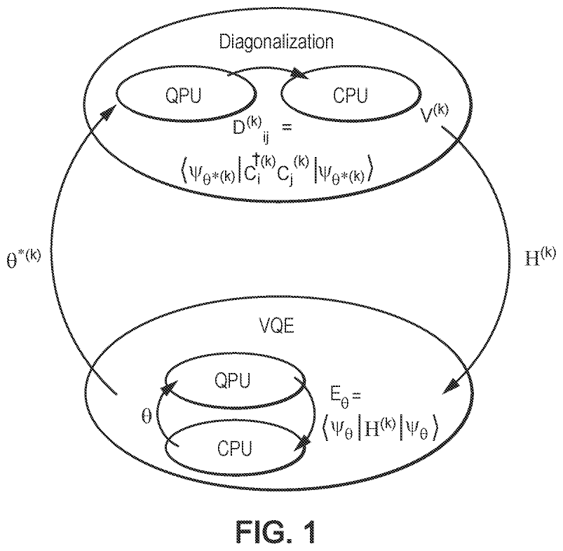

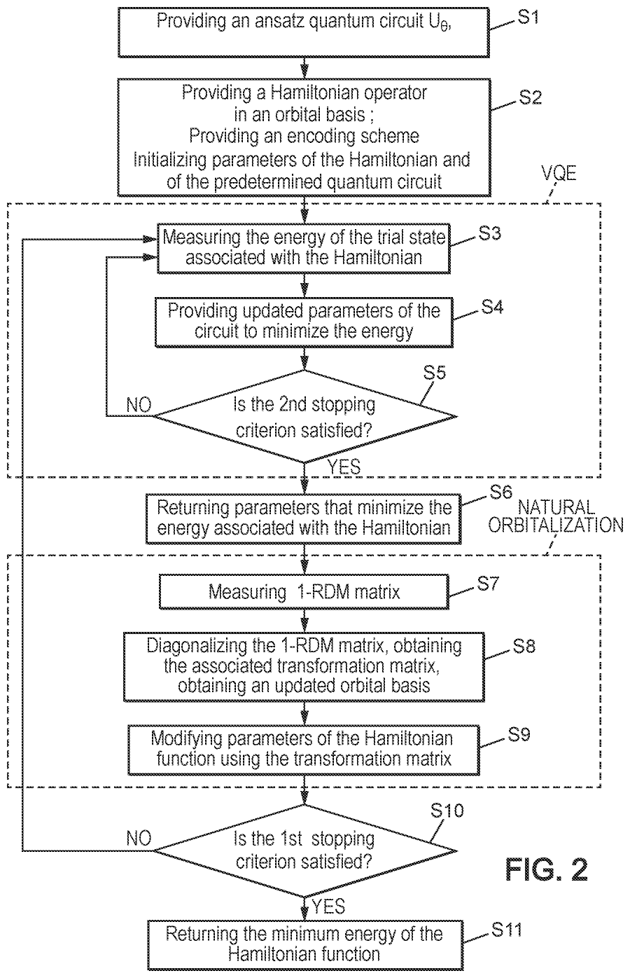

It is now referred to FIGS. 1 and 2 respectively illustrating a flow diagram and a flow chart of the method for finding an optimal quantum state minimizing the energy of a Hamiltonian operator.

At step S1, a predetermined quantum circuit U_{θ}, or simply “quantum circuit U_{θ}” in the following specification, for providing trial quantum states |ψ_{θ} [symbol] is provided. In the field of quantum mechanics, the quantum circuit U_{θ} can be referred to as an ansatz quantum circuit. The quantum circuit U_{θ} comprises at least parametric quantum gates associated with parameters θ to be optimized. The trial quantum states can be expressed as |ψ_{θ} [symbol] =U_{θ}|0 [symbol] ^{⊗M} where |0 [symbol] ^{⊗M} is the initial quantum state of the M-qubit quantum processor.

In an embodiment, the parametric quantum gates of the quantum circuit U_{θ} comprise, for instance, quantum rotation gates, the parameters θ to be optimized comprising, for instance, angles of the quantum rotation gates.

In an embodiment, the method is computed on a Noisy Intermediate Scale Quantum (NISQ) processor, which is a quantum processing unit (QPU), comprising only a few tens of qubits and operating with relatively high error rates. Due to their limited capacity, NISQ processors are generally used in conjunction with classical processors (CPUs), for instance to minimize a cost function. The predetermined quantum circuit should comply with the constraints of the NISQ processor. In particular, the quantum circuit U_{θ} that is used to prepare the trial quantum state |ψ_{θ} [symbol] =U_{θ}|0 [symbol] ^{⊗M} (with M the number of qubits) should fulfill the connectivity and gateset constraints of the quantum processor, and more importantly be compatible with its decoherence properties. On the other hand, the quantum circuit U_{θ} needs to contain enough quantum gates so that the quantum state |ψ_{θ} [symbol] it prepares can approximate with sufficient accuracy the expected quantum ground state, also called the optimal quantum state, to be found.

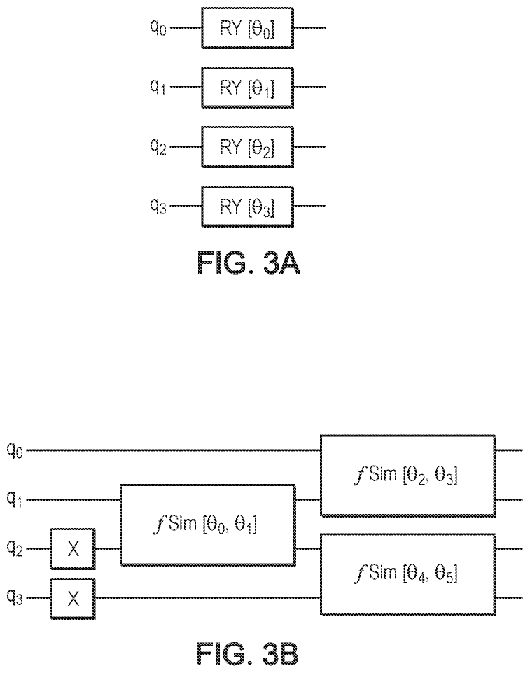

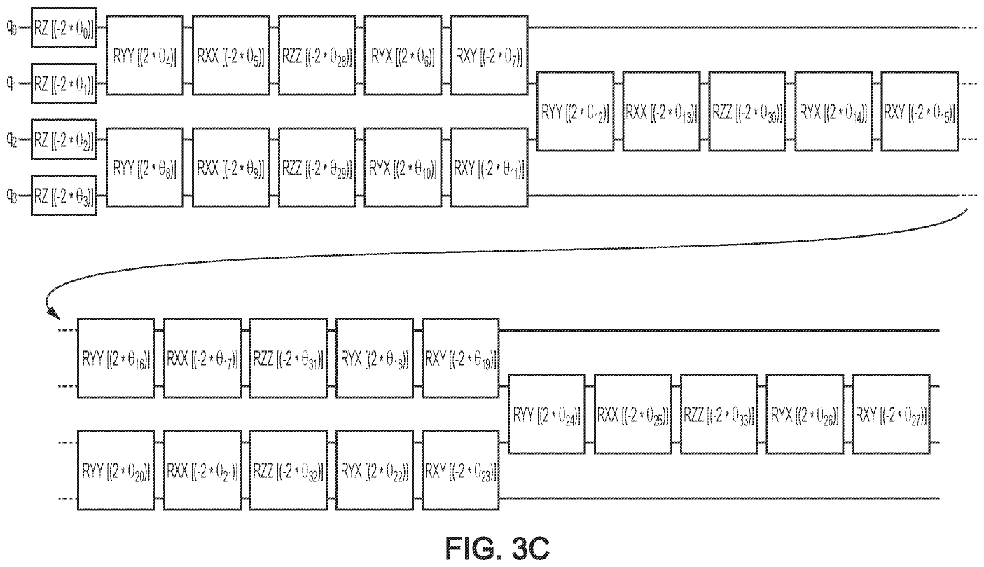

In an embodiment, the quantum circuit U_{θ} may be a “product” quantum circuit, comprising only one-qubit gates in the form of rotations as illustrated on FIG. 3A. In another embodiment, the quantum circuit U_{θ} may be an “fSim” quantum circuit, illustrated on FIG. 3B, and comprising fSim gates developed by the Google© company. In yet another embodiment, the quantum circuit U_{θ} may be the so-called “Low-Depth Circuit Ansatz”, LDCA, circuit.

The product quantum circuit only produces product quantum states, also known as Slater determinants, and is adapted only to non-interacting Hamiltonian operators expressed in their diagonal basis.

The fSim quantum gates of the fSim quantum circuit conserve the number of excitations of a given quantum state.

The LDCA circuit was introduced in the following piece of literature Low-depth circuit ansatz for preparing correlated fermionic states on a quantum computer, Pierre-Luc Dallaire-Demers, Jonathan Romero, Libor Veis, Sukin Sim, Alan Aspuru-Guzik, Jan. 4, 2018.

It should be noted that other types of quantum circuits may be used for preparing the trial quantum states, and the choice of specific type of quantum circuit corresponds to a specific embodiment of the present disclosure.

In an embodiment, the type of quantum gates, and the order of the qubits of the quantum circuit U_{θ} may be fixed, such that a qubit expressed in a scheme is always associated to the same qubit in the hardware, while the quantum circuit U_{θ} comprises at least parametric quantum gates which are tuned by the parameters θ to be optimized.

In another embodiment, the quantum circuit U_{θ} may comprise parametric quantum gates, as well as non-parametric quantum gates.

In yet another embodiment, only some of the parametric quantum gates may be tuned by the parameters θ to be optimized, while some other parametric quantum gates are associated with parameters which may not change during the implementation of the method.

At step S2, the Hamiltonian operator is provided in an orbital basis. In other words, the Hamiltonian operator is expressed in said orbital basis. The orbital basis in which the Hamiltonian operator is initially expressed is referred to as original orbital basis in the sequel.

In an embodiment, the Hamiltonian operator H is a second-quantized Hamiltonian written as:

$$H = {\sum }_{pq} {h}_{pq} {c}_{p}^{†} {c}_{q} + \frac{1}{2} {\sum }_{pqrs} {h}_{pqrs} {c}_{p}^{†} {c}_{q}^{†} {c}_{r} {c}_{s}$$

with p, q, r, sϵM⁴, M being the number of spin-orbitals.

In this embodiment, the Hamiltonian operator may represent the energy of an electronic system, such as a cloud of electrons in the field of an atomic potential in a molecule, with cₚ^{†} and c_{q} the creation and annihilation operators in the original orbital basis ϕₚ(r), h_{pq} the overlap of atomic orbitals of a molecule and h_{pqrs} the collision of two orbitals.

In this embodiment, the original orbital basis in which the Hamiltonian operator is expressed initially is the local orbital basis, i.e. the orbital basis that is centered on the atoms.

A generic cost function may be expressed as the expectation value over a quantum state of some quantum observable.

In this embodiment, the cost function to be minimized is the energy associated with the Hamiltonian operator, which is defined as:

E_{θ}^{(k)}= [symbol] ψ(θ)|H^{(k)|ψ(θ)}

[symbol]

Mathematically, E_{θ}^{(k)} may then be expressed as

$${\sum }_{pq} {h}_{pq} {P}_{pq} + \frac{1}{2} {\sum }_{pqrs} {h}_{pqrs} {P}_{pqrs} ,$$

with P_{pq}= [symbol] ψ(θ)|cₚ^{†}c_{q}|ψ(θ) [symbol] , P_{pqrs}= [symbol] ψ(θ)|cₚ^{†}c_{q}^{†}cᵣcₛ|ψ(θ) [symbol] and where P_{pq} and P_{pqrs} can be obtained from measurements with the quantum processor.

However, the Hamiltonian operator can be expressed differently, depending on the physical or chemical entity to be studied, without modifying the steps of the method for finding an optimal quantum state minimizing the energy associated with a Hamiltonian operator.

In particular, the Hamiltonian operator may be expressed with a form more general than the one used in this embodiment, which may be specific to chemistry and physics problems. A generic Hamiltonian would contain terms of higher order in the creation and annihilation operators cₚ^{†} and c_{q}. Moreover, according to the system to be described with the cost function, the parameters h_{pq} and h_{pqrs} of the Hamiltonian may take different expressions.

Finally, step S2 may also comprise the initialization of the parameters h_{pq} and h_{pqrs} such that h_{pq}^{(k=0)}=h_{pq} and h_{pqrs}^{(k=0)}=h_{pqrs}, with k being the current iteration, for the Hamiltonian operator H^{(k=0)} expressed in the original orbital basis (with cₚ^{†(k=0)}=cₚ^{†} and c_{q}^{(k=0)}=c_{q}).

The order of steps S1 and S2 is purely illustrative.

Steps S3 to S5 are then performed iteratively until a first predefined stopping criterion is met. The first predefined stopping criterion may be satisfied when a maximum number of iterations is performed and/or when the minimum energy associated with the Hamiltonian operator calculated during successive iterations no longer varies.

During steps S3 to S5, the VQE method is applied to find optimized values θ*^{(k)} of the parameters θ that yield an intermediate optimal quantum state which minimizes the energy E_{θ}^{(k)}= [symbol] ψ(θ)|H^{(k)}|ψ(θ) [symbol] associated with the Hamiltonian operator H^{(k)} in the current orbital basis, with |ψ(θ) [symbol] =U(θ)|0 [symbol] .

More specifically, the quantum processor (QPU on FIG. 1, step S3 on FIG. 2) prepares a trial state for the Hamiltonian operator and measures the values P_{pq} and P_{pqrs} representative of the energy associated with the Hamiltonian operator, as defined above. Then the quantum processor transmits these values to a classical processor (CPU on FIG. 1, step S4 on FIG. 2) that determines updated values for the parameters θ for preparing a trial quantum state that may further reduce the energy of the quantum state that is prepared, based on the measurements performed by the quantum processor.

As visible on FIGS. 1 and 2, the VQE method is an iterative scheme which ends when a second predefined stopping criterion is satisfied.

In an embodiment, the second predefined stopping criterion may be satisfied when a maximum number of iterations is performed and/or when the optimized values θ*^{(k)} of the parameters θ updated during successive iterations no longer varies.

At step S6, the VQE method scheme returns the optimized values θ*^{(k)} of the parameters which yield and intermediate optimal quantum state |ψ(θ*^{(k)}) [symbol] minimizing the energy associated with the Hamiltonian operator in the current orbital basis, and transmits it to perform the diagonalization step illustrated on FIG. 1 and FIG. 2.

At step S7, a one particle reduced density matrix Dᵢⱼ^{(k)}, also called 1-RDM, associated to the intermediate optimal quantum state |ψ(θ*^{(k)}) [symbol] is measured on the quantum processor (QPU on FIG. 1) such that Dᵢⱼ^{(k)}(θ*^{(k)})= [symbol] ψ(θ*^{(k)})|cᵢ^{†(k)}cⱼ^{(k)}|ψ(θ*^{(k)}) [symbol] , with θ*^{(k)} the optimized values of the parameters θ yielding the intermediate optimal quantum state |ψ(θ*^{(k)}) [symbol] minimizing the energy associated with the Hamiltonian operator expressed in the current orbital basis, and cᵢ^{†(k)} and cⱼ^{(k)} the creation and annihilation operators in the current orbital basis (i.e. the 1-RDM matrix is measured in the current orbital basis), defined as:

cᵢ^{†(k)}=Σₚ[Vₚᵢ^{(k-1)}]cₚ†^{(k-1)}

cⱼ^{(k)}=Σₚ[Vₚⱼ^{(k-1)}]*cₚ^{(k-1)}

At step S8, the classical processor (CPU on FIG. 1) diagonalizes the 1-RDM to obtain a transformation matrix V^{(k)}. An updated orbital basis is obtained which corresponds to the orbital basis in which the 1-RDM matrix is diagonal.

At step S9, the parameters h_{pq}^{(k)} and h_{pqrs}^{(k)} of the Hamiltonian operator are modified, for instance by the CPU, to h_{pq}^{(k+1)} and h_{pqrs}^{(k+1)} using the transformation matrix V^{(k)} such that:

$$\begin{matrix}{h}_{pq}^{( k + 1 )} = {\sum }_{{p}^{′} {q}^{′}} {V}_{{p}^{′} p}^{( k )} {{h}_{{p}^{′} {q}^{′}}^{( k )} [ {V}^{( k ) †} ]}_{{qq}^{′}} \\ {h}_{pqrs}^{( k + 1 )} = {\sum }_{{p}^{′} {q}^{′} {r}^{′} {s}^{′}} {V}_{{p}^{′} p}^{( k )} {V}_{{q}^{′} q}^{( k )} {{{h}_{{p}^{′} {q}^{′} {r}^{′} {s}^{′}}^{( k )} [ {V}^{( k ) †} ]}_{{rr}^{′}} [ {V}^{( k ) †} ]}_{{ss}^{′}}\end{matrix}$$

The method is then iteratively processed to step S3 with the new parameters h_{pq}^{(k+1)} and h_{pqrs}^{(k+1)} which are used to compute the Hamiltonian operator H^{(k+1)} expressed in the updated orbital basis (wherein the updated orbital basis at the end of iteration k is referred to as current orbital basis at the beginning of iteration k+1), used for the subsequent VQE method iteration, until the first predefined stopping criterion is satisfied (step S11).

When the first predefined stopping criterion (step S11) is satisfied, the minimum value of the energy associated with the Hamiltonian operator is considered to have been found and the optimal quantum state, which corresponds to one of the intermediate quantum states obtained, is returned.

In an embodiment, the optimal quantum state corresponds to the last intermediate optimal quantum state determined.

In another embodiment, the optimal quantum state is the intermediate quantum state which minimizes the most the energy associated with the Hamiltonian, among all the intermediate quantum states obtained during the implementation of the method.

Eventually, by performing the method iteratively, the updated orbital basis, in which the Hamiltonian operator is expressed, will come closer and closer to the natural orbital basis relative to the Hamiltonian's quantum ground state, allowing to reach this quantum ground state while limiting the number of gates in the quantum state preparation circuit. The main property of the natural orbital basis in quantum chemistry is that it is the orbital basis in which the description of a quantum state is the simplest, i.e. the basis that necessitates the least number of Slater determinants to represent the quantum state.

By representing the Hamiltonian operator in an orbital basis that is closer and closer to its natural orbital basis, at each iteration, it results in a higher and higher expressivity of the quantum circuit U_{θ}. In other words, the transformation of the minimization problem to the natural orbital basis endows the method with a guarantee that the problem is placed in the most economical basis, due to the property that the natural orbital basis associated with the Hamiltonian's ground state is the basis in which it can be expressed in a minimal fashion.

As a consequence, the quantum circuit U_{θ} may not perform well in the initial orbital basis but it will perform better and better as the method iteratively converges to the natural orbital basis. Moreover, since the quantum circuit U_{θ} may be shallow, the minimization is robust to noise. Thus, the method allows using shallow quantum circuits, such as a product quantum circuit, or an fSim quantum circuit or a LDCA quantum circuit, as illustrated on FIGS. 3A to 3C.

Mathematically, given a Hamiltonian H and a quantum state |Ψ [symbol] , the natural orbital basis NO is defined as the basis that diagonalizes the one-particle reduced density matrix D_{pq} 1-RDM D_{pq}≡ [symbol] Ψ|cₚ^{†}c_{q}|Ψ [symbol] , with cₚ^{†} and c_{q} the creation and annihilation operators in the original orbital basis ϕₚ(r).

More specifically, if

D_{pq}=V_{pα}n_{α}V_{αq}^{†}, the natural orbitals are defined as \tilde{c}_{α}^{†}≡ΣₚV_{pα}cₚ^{†}.

The main property of the NO basis is that it is the basis where the quantum state |Ψ [symbol] can be represented as a linear combination of the least number of Slater determinants, or, in quantum computing terms, of computational basis quantum states.

These quantum states are defined, in terms of creation operators, as

$$\begin{matrix}{\vert {\tilde{n}}_{1} , {\tilde{n}}_{2} , … , {\tilde{n}}_{M} 〉 = {\prod }_{α = 1}^{M} {( {\tilde{c}}_{α}^{†} )}^{{\tilde{n}}_{α}} \vert 0 〉}^{⊗ M} \\ {\vert {n}_{1} , {n}_{2} , … , {n}_{M} 〉 = {\prod }_{p = 1}^{M} {( {c}_{p}^{†} )}^{{n}_{p}} \vert 0 〉}^{⊗ M}\end{matrix}$$

The quantum state |Ψ [symbol] can be represented either in the original basis, {|n₁, n₂, . . . , n_{M} [symbol] }:

$$\vert Ψ 〉 = {\sum }_{{n}_{1} , {n}_{2} , … , {n}_{M}} {a}_{{n}_{1} , {n}_{2} , … , {n}_{M}} \vert {n}_{1} , {n}_{2} , … , {n}_{M} 〉$$

Or in the natural orbital basis

$$\vert Ψ 〉 = {\sum }_{{\tilde{n}}_{1} , {\tilde{n}}_{2} , … , {\tilde{n}}_{M}} {\tilde{a}}_{{\tilde{n}}_{1} , {\tilde{n}}_{2} , … {\tilde{n}}_{M}} \vert {\tilde{n}}_{1} , {\tilde{n}}_{2} , … , {\tilde{n}}_{M} 〉$$

The above-mentioned property means that the number of nonzero coefficients in this expansion is minimal for the natural orbital basis:

$$\begin{matrix}\# \{ {\tilde{a}}_{{\tilde{n}}_{1} , {\tilde{n}}_{2} , … , {\tilde{n}}_{M}} , \vert {\tilde{a}}_{{\tilde{n}}_{1} , {\tilde{n}}_{2} , … , {\tilde{n}}_{M}} \vert > 0 \} \\ \leq \# \{ {a}_{{n}_{1} , {n}_{2} , … , {n}_{M}} , \vert {a}_{{n}_{1} , {n}_{2} , … , {n}_{M}} \vert > 0 \}\end{matrix}$$

As a consequence, the quantum circuit to prepare the quantum state |Ψ [symbol] is simpler in the natural orbital basis than in the original orbital basis.

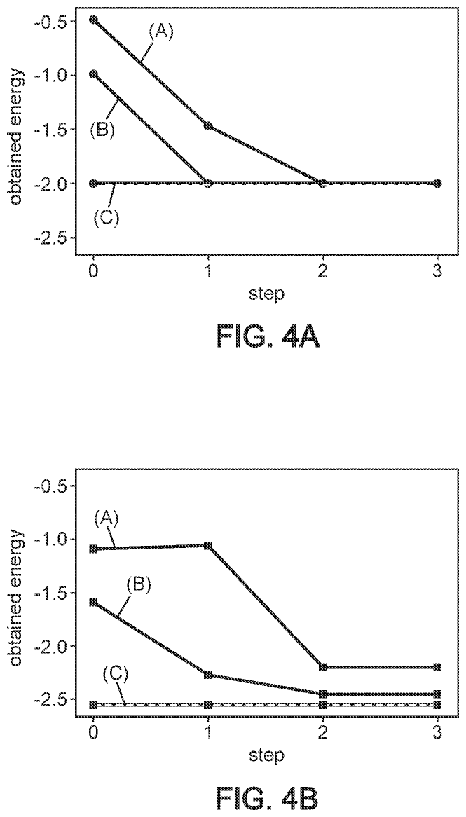

FIGS. 4A to 8B illustrate the performance of the method applied on the Hubbard model at half-filling, with μ=U/2 on an increasing number of sites, the number of sites being 2, 3 and 4. The method has been applied on the three quantum circuits illustrated on FIGS. 3A to 3C.

The Hubbard model is a model used in the field of condensed-matter physics to describe phase transitions in so-called correlated materials, for instance the transition between conducting and insulating systems, such as metals and Mott insulators.

In these specific and non-limiting examples, the method is used to study a chemical compound such as a molecule. The molecule may be a H2, LiH or H20 molecules.

The method further comprises providing an encoding scheme (step S2 of FIG. 2). More precisely, a mapping from physical states defined with fermionic variables to qubit states defined with spin variables is provided.

In an exemplary embodiment, this mapping is achieved through the Jordan-Wigner transformation.

Once the transformation is chosen, the Hamiltonian operator is decomposed accordingly in terms of qubit observables. The quantum circuit U_{θ} is also provided.

In an embodiment, the number of qubits in the quantum circuit U_{θ} is equal to the number of orbitals of the molecule.

In an embodiment, the number of quantum gates in the quantum circuit U_{θ} is also proportional to the number of qubits. For example, if a fSim quantum circuit is used, the number of gates scales as O(Ml), with M the number of qubits in the circuit and 1 the number of fSim layers (l=1 in the non-limiting example provided). If a product quantum circuit is used, the number of quantum gates is M, M being the number of qubits in the circuit. If a LDCA quantum circuit is used, the number of gates scales as O(Ml), with M the number of qubits in the circuit and 1 the number of layers of the LDCA routine employed (l=1 in the example provided).

FIGS. 4A and 4B show the simulated the performance of the method when computed on a noiseless quantum processor.

FIG. 4A illustrates the performance of the method for each of the three quantum circuits, where the line (A) represents the performance for the product circuit, the line (B) represents the fSim circuit and the line (C) represents the LDCA circuit on a Hubbard model for two sites at half-filling where U=0, and where the expected energy value, represented by the dotted line, is approximately equal to −2.0 in this non limiting example.

FIG. 4B illustrates the performance of the method for each of the three quantum circuits, where the line (A) represents the performance for the product circuit, the line (B) represents the fSim circuit and the line (C) represents the LDCA circuit on a Hubbard model for two sites at half-filling where U=1, and where the expected energy value, represented by the dotted line, is approximately equal to −2.5 in this non limiting example.

It can be seen from FIGS. 4A and 4B that for U=0 (FIG. 4A), the product circuit is able to recover the exact energy after a few steps. For U=1 (FIG. 4B), the product circuit does not reach the exact expected energy value because the expected optimal state is entangled due to interactions. However, the energy value reached by the product circuit gets lower as the orbital basis is rotated in a basis closer to the natural orbital basis.

The LDCA circuit reaches the expected energy value for U=0 and U=1 and at each step of the method. This is because the LDCA circuit is very expressive and is numerically able to reach the expected energy value for the size of this circuit.

The fSim circuit is intermediate with respect to the product circuit and LDCA circuit in terms of expressivity. After a few steps, the obtained energy value is closer to the expected energy value as the orbital basis is rotated closer to the natural orbital basis.

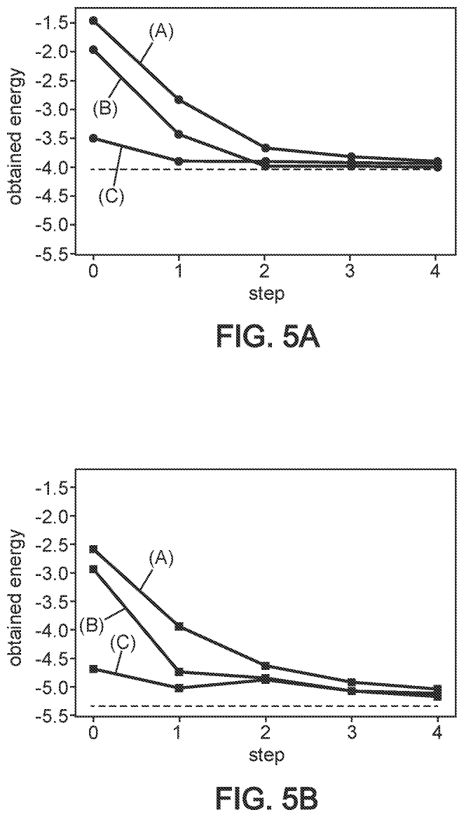

FIGS. 5A and 5B show the performance for the computing of the method on a Hubbard model at half-filling for 4 sites.

FIG. 5A illustrates the performance of the method for each of the three quantum circuits, where the line (A) represents the performance for the product circuit, the line (B) represents the fSim circuit and the line (C) represents the LDCA circuit on a Hubbard model for four sites at half-filling where U=0, and where the expected energy value, represented by the dotted line, is approximately equal to −4.0 in this non limiting example.

FIG. 5B illustrates the performance of the method for each of the three quantum circuits, where the line (A) represents the performance for the product circuit, the line (B) represents the fSim circuit and the line (C) represents the LDCA circuit on a Hubbard model for four sites at half-filling where U=1, and where the expected energy value, represented by the dotted line, is approximately equal to −4.5 in this non limiting example.

The illustrated performances show that the observations made with regards to FIGS. 4A and 4B can be applied to FIGS. 5A and 5B.

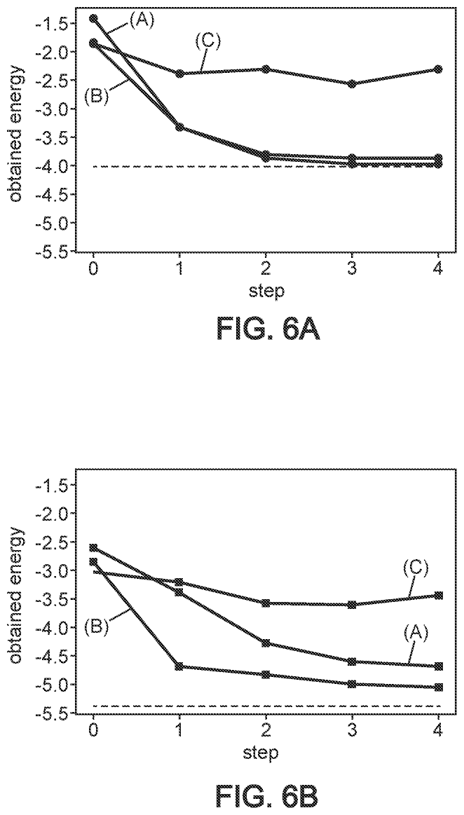

FIGS. 6A and 6B show simulated performance for the computing of the method on a noisy processor.

Since noise is going to penalize long circuits, going to the orbital basis that requires the shortest circuits will be advantageous.

FIG. 6A illustrates the performance of the method for each of the three quantum circuits when computed on a noisy quantum processor, where the line (A) represents the performance for the product circuit, the line (B) represents the fSim circuit and the line (C) represents the LDCA circuit on a Hubbard model for four sites at half-filling where U=0, where the depolarizing noise is equal to 0.001 for the 1-qubit quantum gates and 0.01 for the 2-qubits quantum gates and where the expected energy, represented by the dotted line, value is approximately equal to −4.0 in this non limiting example.

FIG. 6B illustrates the performance of the method for each of the three quantum circuits when computed on a noisy quantum processor, where the line (A) represents the performance for the product circuit, the line (B) represents the fSim circuit and the line (C) represents the LDCA circuit on a Hubbard model for four sites at half-filling where U=1, where the depolarizing noise is of magnitude 0.001 for the 1-qubit quantum gates and 0.01 for the 2-qubits quantum gates and where the expected energy value, represented by the dotted line, is approximately equal to −5.25 in this non limiting example.

It can be seen from FIGS. 6A and 6B that the convergence of the obtained energy to the expected energy value is much faster for the quantum circuits with a small number of parameters such as the product and the fSim quantum circuits.

More precisely, both product and fSim quantum circuits retain their high accuracy at U=0 (FIG. 6A), whereas at U=1, noise slightly degrades their performances. However, due to the large depth of the LDCA circuit, the energy obtained by optimizing the latter in presence of noise is far off the expected energy value.

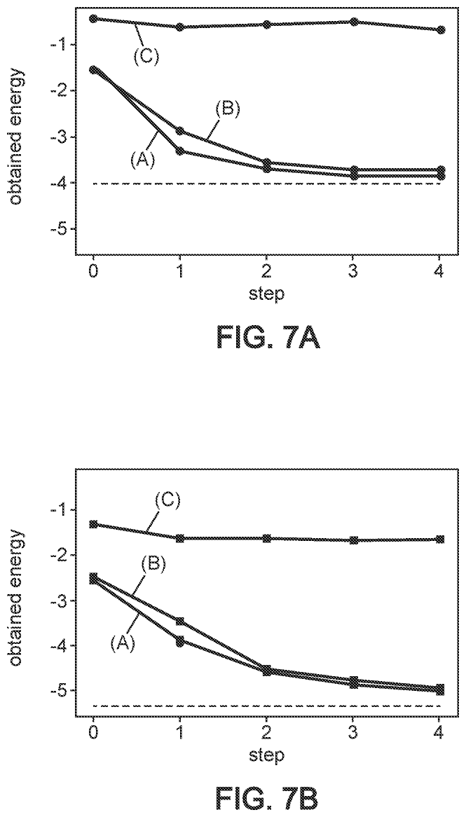

FIGS. 7A and 7B show the performance for the computing of the method on noisier quantum processors.

FIG. 7A illustrates the performance of the method for each of the three quantum circuits when computed on a noisy quantum processor, where the line (A) represents the performance for the product circuit, the line (B) represents the fSim circuit and the line (C) represents the LDCA circuit on a Hubbard model for four sites at half-filling where U=0, where the depolarizing noise is of magnitude0.005 for the 1-qubit quantum gates and 0.05 for the 2-qubits quantum gates and where the expected energy value, represented by the dotted line, is approximately equal to −4.0 in this non limiting example.

FIG. 7B illustrates the performance of the method for each of the three quantum circuits when computed on a noisy quantum processor, where the line (A) represents the performance for the product circuit, the line (B) represents the fSim circuit and the line (C) represents the LDCA circuit on a Hubbard model for four sites at half-filling where U=1, where the depolarizing noise is of magnitude 0.005 for the 1-qubit quantum gates and 0.05 for the 2-qubits quantum gates and where the expected energy value, represented by the dotted line, is approximately equal to −5.25 in this non limiting example.

The observations made with reference to FIGS. 6A and 6B can be applied on the performance illustrated on FIGS. 7A and 7B. More precisely, the accuracy of the obtained energy, compared to the expected energy value, for the product and fSim quantum circuits is maintained, while the energy reached by the LDCA circuit is far off the expected energy value, due to the large depth of the LDCA circuit.

In an embodiment (not shown), the method can be applied for a Hubbard model at half-filling for three sites.

What is claimed is:

1. A method for finding an optimal quantum state minimizing an energy associated with a Hamiltonian operator with a quantum processor and a classical processor by using a Variational Quantum Eigensolver (VQE) method, wherein the Hamiltonian operator represents an energy of a molecule, the quantum processor comprising a predetermined quantum circuit for producing trial quantum states for the Hamiltonian operator, said predetermined quantum circuit comprising at least parametric quantum gates associated with one or more parameters to be optimized, the method comprising:
providing the Hamiltonian operator in an orbital basis and iteratively, until a predefined stopping criterion is satisfied:
performing the VQE method to find optimized values for at least some of the one or more parameters associated with the parametric quantum gates of the predetermined quantum circuit that yield an intermediate optimal quantum state which minimizes the energy associated with the Hamiltonian operator;
computing a one particle reduced density matrix based on the intermediate optimal quantum state;
diagonalizing the one particle reduced density matrix to obtain a transformation matrix, and determining an updated orbital basis in which the one particle reduced density matrix is diagonal, based on the transformation matrix; and
modifying the Hamiltonian operator by using the transformation matrix, to express the Hamiltonian operator in the updated orbital basis;
wherein when the predefined stopping criterion is satisfied, the method further comprises returning, as the optimal quantum state minimizing the energy associated with the Hamiltonian operator, the intermediate optimal quantum state which minimizes the most the energy associated with the Hamiltonian operator.

2. The method according to claim 1, wherein performing the VQE method is an iterative scheme in which the quantum processor is used in conjunction with the classical processor, the quantum processor preparing a trial quantum state for the Hamiltonian operator and performing measurements representative of the energy associated with the Hamiltonian operator for said trial quantum state, and the classical processor updating values of the parameters of the parametric quantum gates of the predetermined quantum circuit based on the measurements performed by the quantum processor, the iterative scheme being executed until a second predefined stopping criterion is satisfied, the VQE method returning the optimized values of the parameters.

3. The method according to claim 1, wherein the predetermined quantum circuit is a product quantum circuit comprising only one-qubit quantum gates in a form of rotations, or a quantum circuit comprising fSim quantum gates or a Low-Depth Circuit Ansatz, LDCA, quantum circuit.

4. The method according to claim 1, wherein the method is applied on a Hubbard model for which the Hamiltonian operator is provided.

5. The method according to claim 4, wherein the Hamiltonian operator is a second-quantized Hamiltonian.

6. The method according to claim 1, wherein a physical quantum state is encoded into a qubit state by means of a Jordan-Wigner transformation from which the Hamiltonian operator is decomposed accordingly in terms of qubit observables.

7. The method according to claim 6, wherein the optimal quantum state corresponding to an eigenvector associated a lowest eigenvalue.

8. The method according to claim 7, wherein the molecule is a H2, LiH and/or H2O molecule.

9. The method according to claim 7, wherein a number of qubits in the predetermined quantum circuit corresponds to a number of spin-orbitals used to describe the molecule.

10. The method according to claim 1, wherein the predefined stopping criterion is a maximum number of iterations and/or a minimum change of a variance between the minimums of the energy associated with the Hamiltonian operator obtained after two consecutive iterations.

11. The method according to claim 1, wherein the parametric quantum gates comprise rotation quantum gates, and wherein the parameters associated with said rotation quantum gates comprise values of angles.

12. A method for determining a quantum state of a chemical compound, comprising:
a method for finding an optimal quantum state minimizing the energy associated with a Hamiltonian operator with a quantum processor and a classical processor according to claim 1,
wherein an expectation value of the Hamiltonian operator over a given quantum state corresponds to the energy of said quantum state.

13. A method for determining physical properties of materials comprising:
a method for finding an optimal quantum state by minimizing the energy associated with a Hamiltonian operator with a quantum processor and a classical processor according to claim 1.
